(19) United States
(12) Patent Application Publication
(10) Pub. No.: US 2010/0185000 A1
(43) Pub. Date: Jul. 22, 2010

(54) TITANIUM COMPOUND AND PROCESS FOR ASYMMETRIC CYANATION OF IMINES

(75) Inventors: Abdul Majeed Seayad, Parc Oasis (SG); Christina Chai, Jurong Island (SG); Balamurugan Ramalingam, Singapore (SG); Takushi Nagata, Jurong Island (SG); Kazuhiko Yoshinaga, Jurong Island (SG)

Correspondence Address: WOLF GREENFIELD & SACKS, P.C., 600 ATLANTIC AVENUE, BOSTON, MA, 02210-2206, US

(73) Assignees: Agency for Science, Technology and Research, Connexis (SG); Mitsui Chemicals Inc., Tokyo (JP)

(21) Appl. No.: 12/593,643

(22) PCT Filed: Sep. 28, 2007
(86) PCT No.: PCT/SG07/00326

§ 371 Date: Mar. 22, 2010

(30) Foreign Application Priority Data
Mar. 29, 2007 (SG) PCT/SG2007/000084

(51) Int. Cl. C07C 253/30; C07F 7/28; C07D 307/54

(52) U.S. Cl. 549/492; 556/51; 558/390; 558/404

(57) ABSTRACT

The present invention relates to titanium catalysts for asymmetric synthesis reactions produced by bringing a reaction mixture obtained by contacting water and a titanium alkoxide into contact with an optically active ligand represented by the general formula (a), wherein R¹, R², R³, and R⁴ are independently a hydrogen atom, an alkyl group, or the like, and A* represents a group with two or more carbon atoms having an asymmetric carbon atom or axial asymmetry. The invention further relates to a process for asymmetric cyanation of imines, wherein the process comprises reacting an imine with a cyanating agent in the presence of the titanium catalyst.

### FIELD OF THE INVENTION

The present invention relates to a titanium compound and a process for producing optically active alpha-aminonitriles according to the asymmetric cyanation reaction of an imine using such a titanium compound. The optically active alpha-aminonitriles are useful as intermediates in the synthesis of pharmaceuticals and fine-chemicals.

### BACKGROUND OF THE INVENTION

One of the oldest, most efficient and economic methods of synthesizing alpha-amino acids is the use of a three component Strecker reaction of aldehydes or ketones with ammonia (or an equivalent) in the presence of a cyanide source. Subsequent hydrolysis of the resultant aminonitrile yields the corresponding alpha-amino acids, as shown by the reaction in FIG. 1A. FIG. 1B shows a modified Strecker reaction, a popular and widely used alternative route for synthesizing alpha-amino acids, wherein an amine is used instead of ammonia and pre-formation of imines is followed by hydrocyanation.

Despite the efficiency and versatility of the Strecker reaction, no catalytic asymmetric version of the reaction or catalytic asymmetric hydrocyanation of imines was reported until the mid-1990s. Since then there has been considerable advances in the development of efficient asymmetric processes for the synthesis of optically active alpha-amino acids, especially nonproteinogenic alpha-amino acids. Both organometallic- and organo-catalysts have also been used in the asymmetric hydrocyanation of imines to produce the corresponding chiral alpha-aminonitriles in the presence of a suitable cyanide source. Although good to excellent results have been reported, many of these catalyst systems utilize expensive ligands and catalysts that are prepared through multi-step synthesis, as well as rigorous conditions such as low temperatures.

Accordingly, improved compound and methods are needed.

### SUMMARY OF THE INVENTION

The present invention provides titanium catalysts for asymmetric synthesis reactions, produced by bringing a reaction mixture obtained by contacting water and a titanium alkoxide into contact with an optically active ligand represented by the general formula (a),

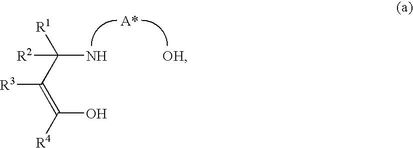

wherein R¹, R², R³, and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may have a substituent, or two or more of R¹, R², R³, and R⁴ may be linked together to form a ring, and the ring may have a substituent; and A* represents a group with two or more carbon atoms having an asymmetric carbon atom or axial asymmetry.

In some embodiments, the optically active ligand represented by said general formula (a) may be represented by general formula (b),

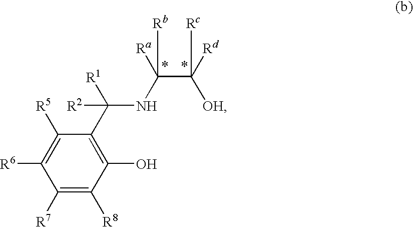

wherein R^{a}, R^{b}, R^{c}, and R^{d} are each a hydrogen atom, an alkyl group, an aryl group, alkoxycarbonyl group, an aryloxycarbonyl group or an aminocarbonyl group, each of which may have a substituent, or two or more of R^{a}, R^{b}, R^{c}, and R^{d} may be linked together to form a ring, and the ring may have a substituent; at least one of R^{a}, R^{b}, R^{c}, and R^{d} is a different group; both or at least one of the carbon atoms indicated as * become an asymmetric center; and parts indicated as (NH) and (OH) do not belong to A*, and represent an amino group and a hydroxyl group, respectively, corresponding to those in said general formula (a) to which A* is bonded; R⁵, R⁶, R⁷, and R⁸ are independently a hydrogen atom, a halogen atom, an alkyl group, an alkenyl group, an aryl group, an aromatic heterocyclic group, a non-aromatic heterocyclic group, an alkoxycarbonyl group, an aryloxycarbonyl group, a hydroxyl group, an alkoxy group, an aryloxy group, an amino group, a cyano group, a nitro group, a silyl group or a siloxy group which may have a substituent, each of which may be linked together to form a ring.

The present invention also provides processes for asymmetric cyanation of imines, comprising reacting an imine with a cyanating agent in the presence of a titanium catalyst of the invention. In some embodiments, the imine is represented by the general formula (c),

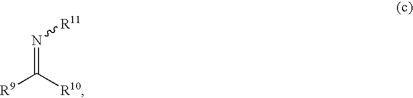

wherein R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰; R⁹ and R¹⁰ may be linked together to form a ring, and the ring may have a substituent; R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl, or sulfoxy group, each of which may have a substituent; and R¹¹ may be linked either to R⁹ or R¹⁰ to form a ring through a carbon chain, and the ring may have substituents.

Processes for asymmetric cyanation of imines may comprise reacting an imine and a cyanating agent in the presence of a catalyst to form an optically active alpha-aminonitrile, wherein the catalyst is present in an amount from about 0.5 to 30 mol %, relative to the imine, and comprises a product of interaction between a titanium alkoxide precatalyst (e.g., a partially hydrolyzed titanium alkoxide precatalyst prepared by contacting water with titanium alkoxide monomer) and an optically active compound having the ability to ligate the titanium. In some embodiments, the catalyst is present in an amount from about 1 to 30 mol %, relative to the imine. In some embodiments, the catalyst is present in an amount less than 10 mol % (e.g., from 2.5 to 5.0 mol %), relative to the imine. The process may be conducted at any temperature and with any reaction time suited for a particular application. In some embodiments, the process is conducted at a reaction temperature between −78° C. and 80° C. In some embodiments, the process may comprise reacting an imine and a cyanating agent in the presence of a catalyst at a temperature greater than 0° C. and/or with a reaction time of less than six hours, or less than two hours, and with a yield of at least 50%, or, in some cases, with high to quantitative yields, and wherein the optically active alpha-aminonitrile is obtained in good to excellent enantiomeric excess (e.g., at least 90%).

### BRIEF DESCRIPTION OF THE DRAWINGS

FIG. 1A shows the synthesis a an alpha-amino acid via a Strecker reaction and subsequent hydrolysis of the resultant aminonitrile.

FIG. 1B shows the synthesis of an alpha-amino acid modified Strecker reaction and subsequent hydrolysis of the resultant aminonitrile.

FIG. 2 shows the asymmetric cyanation of N-benzylbenzylidineamine in the presence of an optically active titanium catalyst of the invention and trimethylsilyl cyanide, according to one embodiment of the invention.

FIG. 3 shows a one-pot synthesis of an optically active alpha-aminonitrile, according to one embodiment of the invention.

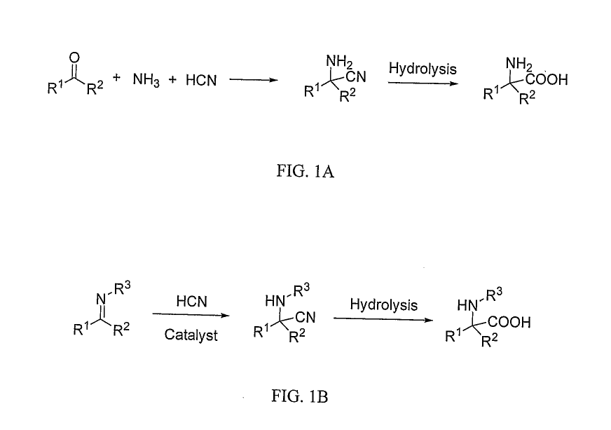

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention relates to a titanium compound and a process for producing optically active alpha-aminonitriles according to the asymmetric cyanation reaction of an imine using such a titanium compound.

Compounds (e.g., catalysts) and methods of the invention involve titanium catalysts useful for asymmetric synthesis reactions, including carbon-carbon bond forming reactions. In some embodiments, the present invention provides catalysts and related methods for asymmetric Strecker-type reactions, such as the asymmetric cyanation of imines for the synthesis of optically active alpha-aminonitriles. The present invention provides efficient catalysts based on inexpensive, stable ligands derived from readily available building blocks. Catalysts and methods of the invention that may advantageously be used under mild reaction conditions, such as room temperature and/or under ambient conditions, to achieve high yields (e.g., >99%) and excellent enantioselectivities (e.g., >90%, >95%, >98%).

The present invention relates to the discovery that optically active alpha-aminonitriles may be produced in high yield and with high optical purity using an efficient catalyst and related methods involving lower amounts of catalyst and shorter reaction times relative to previous methods. Optically active alpha-aminonitriles are useful intermediates in the synthesis of pharmaceuticals, fine chemicals, and the like. In some embodiments, optically active alpha-aminonitriles are useful intermediates in the synthesis of alpha-amino acids. In a particular set of embodiments, the invention relates to the asymmetric cyanation of imines for the synthesis of optically active alpha-aminonitriles using a partially hydrolyzed titanium-alkoxide catalyst system in the presence of an optically active ligand such as tridentate N-salicyl-beta-aminoalcohol, for example. As described herein, the present invention provides titanium catalysts for asymmetric synthesis reactions. The titanium catalyst may be produced by combining water or a water source with a titanium alkoxide to form a reaction mixture, which may then be brought into contact with an optically active ligand.

The following terms refer to any groups mentioned in the present invention unless otherwise indicated.

The term “alkyl group” refers to a linear, branched or cyclic alkyl group having 1 to 20 carbon atoms. In one embodiment of the present invention, the alkyl group may have 1 to 15 carbon atoms, for example 1 to 10 carbon atoms. Examples of linear alkyl groups may include, but are not limited to, a methyl group, an ethyl group, a n-propyl group, a n-butyl group, a n-pentyl group, a n-hexyl group, a n-heptyl group, a n-octyl group, a nonyl group, a n-decyl group and the like. Examples of branched alkyl groups may include, but are not limited to, an isopropyl group, an isobutyl group, sec-butyl group, a tert-butyl group, a 2-pentyl group, a 3-pentyl group, an isopentyl group, a neopentyl group, an amyl group and the like. Examples of cyclic alkyl groups may be, but are not limited to, a cyclopropyl group, a cyclobutyl group, a cyclopentyl group, a cyclohexyl group, a cycloheptyl group, a cycloctyl group and the like.

The term “alkenyl group” refers to a linear, branched or cyclic alkenyl group having 2 to 20 carbon atoms, for example 1 to 10 carbon atoms, wherein at least one carbon-carbon double bond is present. Examples of an alkenyl group may include, but are not limited to, a vinyl group, an allyl group, a crotyl group, a cyclohexenyl group, an isopropenyl group and the like.

The term “alkynyl group” refers to an alkynyl group having 2 to 20 carbon atoms, for example 2 to 10 carbon atoms, wherein at least one carbon-carbon triple bond is present. Examples may include, but are not limited to, an ethynyl group, a 1-propynyl group, a 2-propynyl group, a 1-butynyl group, a 1-pentynyl group and the like.

The term “alkoxy” refers to a linear, branched or cyclic alkoxy group having 1 to 20 carbon atoms, for example 1 to 10 carbon atoms, wherein an alkyl group is bonded to a negatively charged oxygen atom. Examples may include, but are not limited to, a methoxy group, an ethoxy group, a n-propoxy group, an isopropoxy group, a n-butoxy group, a cyclopentyloxy group, a cyclohexyloxy group, a menthyloxy group and the like.

The term “aryl group” refers to an aryl group referring to any functional group or substituent derived from a simple aromatic ring having 6 to 20 carbon atoms. In one embodiment of the present invention, the aryl group may have 6 to 10 carbon atoms. Examples may include, but are not limited to, a phenyl group, a napththyl group, a biphenyl group, an anthryl group and the like.

The term “aryloxy group” refers to an aryloxy group having 6 to 20 carbon atoms, for example 6 to 10 carbon atoms, wherein an aryl group is bonded to a negatively charged oxygen atom. Examples may include, but are not limited to, a phenoxy group, a naphthyloxy group and the like.

The term “aromatic heterocyclic group” refers to an aromatic heterocyclic group having 3 to 20 carbon atoms, for example 1 to 10 carbon atoms, wherein at least one carbon atom of the aromatic group is replaced by a heteroatom such as nitrogen, oxygen or sulfur. Examples may include, but are not limited to, an imidazolyl group, a furyl group, a thienyl group, a pyridyl group and the like.

The term “non-aromatic heterocyclic group” refers to a non-aromatic heterocyclic group having 4 to 20 carbon atoms, for example 4 to 10 carbon atoms, wherein at least one carbon atom of the non-aromatic group is replaced by a heteroatom such as nitrogen, oxygen or sulfur. Examples may include, but are not limited to, a pyrrolidyl group, a piperidyl group, a tetrahydrofuryl group and the like.

The term “acyl group” refers to an alkylcarbonyl group having 2 to 20 carbon atoms, for example 1 to 10 carbon atoms and an arylcarbonyl group having 6 to 20 carbon atoms, for example 1 to 10 carbon atoms.

The term “alkylcarbonyl group” refers to, but is not limited to, an acetyl group, a propionyl group, a butyryl group, an isobutyryl group, a pivaloyl group and the like.

The term “arylcarbonyl group” refers to, but is not limited to, a benzoyl group, a napththoyl group, a anthrylcarbonyl group and the like.

The term “alkoxycarbonyl group” refers to a linear, branched or cyclic alkoxycarbonyl group having 2 to 20 carbon atoms, for example 2 to 10 carbon atoms. Examples may include, but are not limited to, a methoxycarbonyl group, an ethoxycarbonyl group, a n-butoxycarbonyl group, a n-octyloxycarbonyl group, an isopropoxycarbonyl group, a tert-butoxycarbonyl group, a cyclopentyloxycarbonyl group, a cyclohexyloxycarbonyl group, a cyclooctyloxycarbonyl group, an L-menthyloxycarbonyl group, a D-menthyloxycarbonyl group and the like.

The term “aryloxycarbonyl group” refers to an aryloxycarbonyl group having 7 to 20 carbon atoms, for example 7 to 15 carbon atoms. Examples may include, but are not limited to, a phenoxycarbonyl group, alpha-naphthyloxycarbonyl group and the like.

The term “aminocarbonyl group” refers to an aminocarbonyl group having a hydrogen atom, an alkyl group, an aryl group, and two of the substituents other than a carbonyl group to be bonded to a nitrogen atom may be linked together to form a ring. Examples may include, but are not limited to an isopropylaminocarbonyl group, a cyclohexylaminocarbonyl group, a tert-butylaminocarbonyl group, a tert-amylaminocarbonyl group, a dimethylaminocarbonyl group, a diethylaminocarbonyl group, diisopropylaminocarbonyl group, a diisobutylaminocarbonyl group, a dicyclohexylaminocarbonyl group, a tert-butylisopropylaminocarbonyl group, a phenylaminocarbonyl group, a pyrrolidylcarbonyl group, a piperidylcarbonyl group, an indolecarbonyl group and the like.

The term “amino group” refers to organic compounds and a type of functional group that contain nitrogen as the key atom. The term refers to an amino group having a hydrogen atom, a linear, branched or cyclic alkyl group, or an amino group having an aryl group. Two substituents to be bonded to a nitrogen atom may be linked together to form a ring. Examples of the amino group having an alkyl group or an aryl group may include, but are not limited to, an isopropylamino group, a cyclohexylamino group, a tert-butylamino group, a tert-amylamino group, a dimethylamino group, a diethylamino group, a diisopropylamino group, a diisobutylamino group, a dicyclohexylamino group, a tert-butylisopropylamino group, a pyrrolidyl group, a piperidyl group, an indole group and the like.

The term “halogen atom” refers to F, Cl, Br, I, and the like.

The term “silyl group” refers to a silyl group having 2 to 20 carbon atoms, wherein the silyl group can be considered as silicon analogue of an alkyl. Examples may include, but are not limited to, a trimethylsilyl group, a tert-butyldimethylsilyl group and the like.

The term “siloxy group” refers to a siloxy group having 2 to 20 carbon atoms. Examples may include, but are not limited to, a trimethylsiloxy group, a tert-butyldimethylsiloxy group, a tert-butyldiphenylsiloxy group and the like.

All of the above mentioned groups may optionally have one or more substituents. “Having one or more substituents” in the context of the present invention means that at least one hydrogen atom of the above compounds may be replaced by F, Cl, Br, I, OH, CN, NO₂, NH₂, SO₂, an alkyl group, an aryl group, an aromatic heterocyclic group, a non-aromatic heterocyclic group, an oxygen containing group, a nitrogen containing group, a silicon containing group or the like.

Examples of the oxygen containing group may include, but are not limited to, those having 1 to 20 carbon atoms such as an alkoxy group, an aryloxy group, an alkoxycarbonyl group, a aryloxycarbonyl group, an acyloxy group and the like. Examples of the nitrogen containing group may include, but are not limited to, an amino group having 1 to 20 carbon atoms, an amide group having 1 to 20 carbon atoms, a nitro group, a cyano group and the like. Examples of the silicon containing group may include, but are not limited to, those having 1 to 20 carbon atoms such as a silyl group, a silyloxy group and the like.

Examples of substituted alkyl groups may include, but are not limited to, a chloromethyl group, a 2-chloroethyl group, a trifluoromethyl group, a 2,2,2-trifluoroethyl group, a perfluoroethyl group, a perfluorohexyl, a substituted or unsubstituted aralkyl group such as a benzyl group, a diphenylmethyl group, a trityl group, a 4-methoxybenzyl group, a 2-phenylethyl group, a cumyl group, an alpha-napthylmethyl, a 2-pyridylmethyl group, a 2-furfuryl group, a 3-furfuryl group, a 2-thienylmethyl group, a 2-tetrahydrofurfuryl group, a 3-tetrahydrofurfuryl group, a methoxymethyl group, a methoxyethyl group, a phenoxyethyl group, an isopropoxymethyl group, a tert-butoxymethyl group, a cyclohexyloxymethyl group, a L-menthyloxymethyl group, a D-menthyloxymethyl group, a phenoxymethyl group, a benzyloxymethyl group, a phenoxymethyl group, an acetyloxymethyl group, a 2,4,6-trimethylbenzoyloxymethyl, a 2-(dimethylamino)ethyl group, a 3-(diphenylamino)propyl group, a 2-(trimethylsiloxy)ethyl group and the like.

Examples of substituted alkenyl groups may include, but are not limited to, a 2-chlorovinyl group, a 2,2-dichlorovinyl group, a 3-chloroisopropenyl group and the like.

Examples of substituted alkynyl groups may include, but are not limited to, a 3-chloro-1-propynyl group, a 2-phenylethynyl group, a 3-phenyl-2-propynyl group, a 2-(2-pyridylethynyl) group, a 2-tetrahydrofurylethynyl group, a 2-methoxyethynyl group, a 2-phenoxyethynyl group, a 2-(dimethylamino)ethynyl group, a 3-(diphenylamino)propynyl group, a 2-(trimethylsiloxy)ethynyl group and the like.

Examples of substituted alkoxy groups may include, but are not limited to, a 2,2,2-trifluoroethoxy group, a benzyloxy group, a 4-methoxybenzyloxy group, a 2-phenylethoxy group, a 2-pyridylmethoxy group, a furfuryloxy group, a 2-thienylmethoxy group, a tetahydrofurfuryloxy group and the like.

Examples of substituted aryl groups may include, but are not limited to, a 4-fluorophenyl group, a pentafluorophenyl group, a tolyl group, a dimethylphenyl group such as a 3,5-dimethylphenyl group, a 2,4,6-trimethylphenyl group, a 4-isopropylphenyl group, a 3,5-diisopropylphenyl group, a 2,6-diisopropylphenyl group, a 4-tert-butylphenyl group, a 2,6-di-tert-butylphenyl group, a 4-methoxyphenyl group, a 3,5-dimethoxyphenyl group, a 3,5-diisopropoxyphenyl group, a 2,4,6-triisopropoxyphenyl group, a 2,6-diphenoxyphenyl group, a 4-(dimethylamino)phenyl group, a 4-nitrophenyl group, 3,5-bis(trimethylsilyl)phenyl group, a 3,5-bis(trimethylsiloxy)phenyl group and the like.

Examples of substituted aryloxy groups may include, but are not limited to, a pentafluorophenoxy group, a 2,6-dimethylphenoxy group, a 2,4,6-trimethylphenoxy group, a 2,6-dimethoxyphenoxy group, a 2,6-diisopropoxyphenoxy group, a 4-(dimethylamino)phenoxy group, a 4-cyanophenoxy group, a 2,6 bis(trimethylsilyl)phenoxy group, a 2,6-bis(trimethylsiloxy)phenoxy group and the like.

Examples of substituted aromatic heterocyclic groups may include, but are not limited to, an N-methylimidazolyl group, a 4,5-dimethyl-2-furyl group, a 5-butoxycarbonyl-2-furyl group, a 5-butylaminocarbonyl-2-furyl group, and the like.

Examples of substituted non-aromatic heterocyclic groups may include, but are not limited to, a 3-methyl-2-tetrahydrofuranyl group, a N-phenyl-4-piperidyl group, a 3-methoxy-2-pyrrolidyl group and the like.

Examples of substituted alkylcarbonyl group may include, but are not limited to, a trifluoroacetyl group and the like.

Examples of substituted arylcarbonyl groups may include, but are not limited to, a pentafluorobenzoyl group, a 3,5-dimethylbenzoyl group, a 2,4,6-trimethylbenzoyl group, a 2,6-dimethoxybenzoyl group, a 2,6-diisopropoxybenzoyl group, a 4-(dimethylamino)benzoyl group, a 4-cyanobenzoyl group, a 2,6-bis(trimethylsilyl)benzoyl group, a 2,6-bis(trimethylsiloxy)benzoyl group and the like.

Examples of the alkoxycarbonyl group having a halogen atom include a 2,2,2-trifluoroethoxycarbonyl group, a benzyloxycarbonyl group, a 4-methoxybenzyloxycarbonyl group, a 2-phenylethoxycarbonyl group, a cumyloxycarbonyl group, an alpha-naphthylmethoxycarbonyl group, a 2-pyridylmethoxycarbonyl group, a furfuryloxycarbonyl group, a 2-thienylmethoxycarbonyl group, a tetrahydrofurfuryloxycarbonyl group, and the like.

Examples of substituted aryloxycarbonyl groups may include, but are not limited to, a pentafluorophenoxycarbonyl group, a 2,6-dimethylphenoxycarbonyl group, a 2,4,6-trimethylphenoxycarbonyl group, a 2,6-dimethoxyphenoxycarbonyl group, a 2,6-diisopropoxyphenoxycarbonyl group, a 4-(dimethylamino)phenoxycarbonyl group, a 4-cyanophenoxycarbonyl group, a 2,6-bis(trimethylsilyl)phenoxycarbonyl group, a 2,6-bis(trimethylsiloxy)phenoxycarbonyl group and the like.

Examples of substituted aminocarbonyl groups may include, but are not limited to, a 2-chloroethylaminocarbonyl group, a perfluoroethylaminocarbonyl group, a 4-chlorophenylaminocarbonyl group, a pentafluorophenylaminocarbonyl group, a benzylaminocarbonyl group, a 2-phenylethylaminocarbonyl group, an alpha-naphthylmethylaminocarbonyl and a 2,4,6-trimethylphenylaminocarbonyl group and the like.

Examples of substituted amino groups may include, but are not limited to, a 2,2,2-trichloroethylamino group, a perfluoroethylamino group, a pentafluorophenylamino group, a benzylamino group, a 2-phenylethylamino group, an alpha-naphthylmethylamino and a 2,4,6-trimethylphenylamino group and the like.

In one aspect, the present invention relates to titanium catalysts for asymmetric synthesis reactions, such as asymmetric cyanation of imines. The titanium catalyst may be produced by contacting a reaction mixture comprising a titanium alkoxide with an optically active ligand. The reaction mixture comprising the titanium alkoxide may be obtained by combining water, a titanium alkoxide, and optionally additional components, such as solvents, hydrolyzing agents, additives, and the like. In some embodiments, the titanium alkoxide may be in monomeric form in the absence of water, and, upon contact with water, a partially hydrolyzed titanium alkoxide species may be produced, i.e., a “precatalyst.” As used herein, a “precatalyst” may refer to a chemical species which, upon activation, may produce an active catalyst species in a reaction. For example, the partially hydrolyzed titanium alkoxide precatalyst may be combined with the optically active ligand to form the catalyst. As used herein, the term “catalyst” includes active forms of the catalyst participating in the reaction as well as catalyst precursors (e.g., precatalysts) that may be converted in situ into the active form of the catalyst.

In some embodiments, the titanium alkoxide used in the preparation of the titanium catalyst may be a compound represented by the general formula (d),

Ti(OR′)₄ (d)

wherein R′ is an alkyl group or an aryl group, each of which may have a substituent. In some embodiments, R′ is an alkyl group, such as ethyl, n-butyl, n-propyl, iso-propyl, and the like. For example, the titanium alkoxide used may be Ti(OMe)₄, Ti(OEt)₄, Ti(On-Pr)₄, Ti(Oi-Pr)₄, or Ti(On-Bu)₄. In some embodiments, R′ is an aryl group.

The titanium compound (e.g., catalyst) of the present invention may be produced from a reaction mixture of a partially hydrolyzed titanium alkoxide obtained by contacting water with a titanium alkoxide monomer, and an optically active ligand represented by the general formula (a),

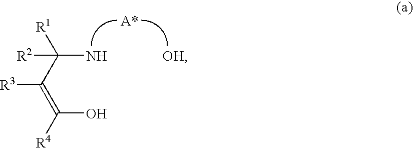

wherein R¹, R², R³, and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may have a substituent, or two or more of R¹, R², R³, and R⁴ may be linked together to form a ring, and the ring may have a substituent; and A* represents a group with two or more carbon atoms having an asymmetric carbon atom or axial asymmetry.

In some cases, R¹, R², R³, or R⁴ may be an alkyl group, optionally having one or more substituents. Furthermore, two or more of R¹, R², R³, and R⁴ may be linked together to form a ring. The ring may be an aliphatic or aromatic hydrocarbon ring. The formed rings may be condensed to form a ring, respectively. In some embodiments, the aliphatic hydrocarbon ring is a 10- or less-membered ring, such as a 3- to 7-membered ring, or a 5- or 6-membered ring. The aliphatic hydrocarbon ring may have unsaturated bonds. The aromatic hydrocarbon ring may be a 6-membered ring, such as a phenyl ring. For example, when two of R¹, R², R³, and R⁴ are linked together to form —(CH₂)₄— or —CH═CH—CH═CH—, a cyclohexene ring (included in the aliphatic hydrocarbon ring) or a phenyl ring (included in the aromatic hydrocarbon ring) may be formed, respectively. The ring may have one or more substituents, including a halogen atom, an alkyl group, an aryl group, an alkoxy group, an aryloxy group, an amino group, a nitro group, a cyano group, a silyl group and a silyloxy group, and the like.

In one set of embodiments, R¹ and R² are hydrogen atoms, and R³ and R⁴ are linked together to form a phenyl ring, wherein the phenyl ring may have one or more substituents.

In the general formula (a), A* represents an optically active group with two or more carbon atoms, and preferably 2 to 40 carbon atoms, having an asymmetric carbon atom or axial asymmetry which may have a substituent. Examples of A* include the following structures,

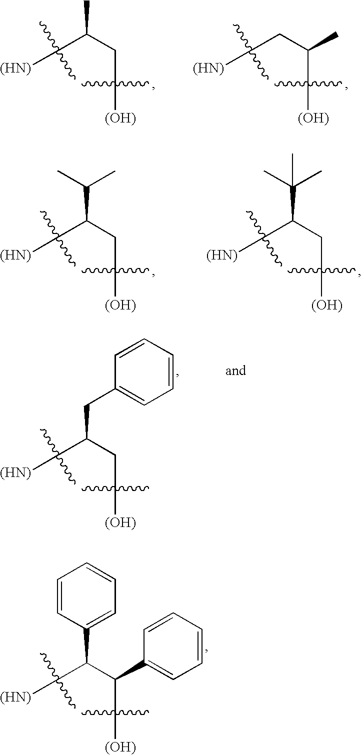

wherein parts indicated as (N) and (OH) do not belong to A*, and represent an amino group and a hydroxyl group, respectively, corresponding to those in the above general formula (a) to which A* is bonded.

In some cases, the optically active ligand is represented by the general formula (b),

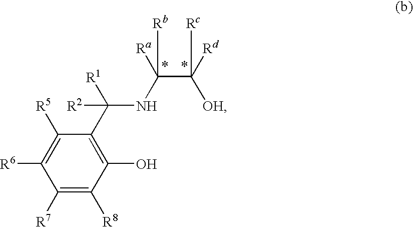

wherein R^{a}, R^{b}, R^{c}, and R^{d} are each a hydrogen atom, an alkyl group, an aryl group, alkoxycarbonyl group, an aryloxycarbonyl group or an aminocarbonyl group, each of which may have a substituent, or two or more of R^{a}, R^{b}, R^{c}, and R^{d} may be linked together to form a ring, and the ring may have a substituent; at least one of R^{a}, R^{b}, R^{c}, and R^{d} is a different group; both or at least one of the carbon atoms indicated as * become an asymmetric center; and parts indicated as (NH) and (OH) do not belong to A*, and represent an amino group and a hydroxyl group, respectively, corresponding to those in said general formula (a) to which A* is bonded; R⁵, R⁶, R⁷, and R⁸ are independently a hydrogen atom, a halogen atom, an alkyl group, an alkenyl group, an aryl group, an aromatic heterocyclic group, a non-aromatic heterocyclic group, an alkoxycarbonyl group, an aryloxycarbonyl group, a hydroxyl group, an alkoxy group, an aryloxy group, an amino group, a cyano group, a nitro group, a silyl group or a siloxy group which may have a substituent, each of which may be linked together to form a ring.

In some cases, R^{a} is methyl, ethyl, n-propyl, iso-propyl, n-butyl, iso-butyl, sec-butyl, tert-butyl, or benzyl, and R^{b}, R^{c}, and R^{d} are hydrogen atoms.

Examples of the optically active ligand include, but are not limited to,

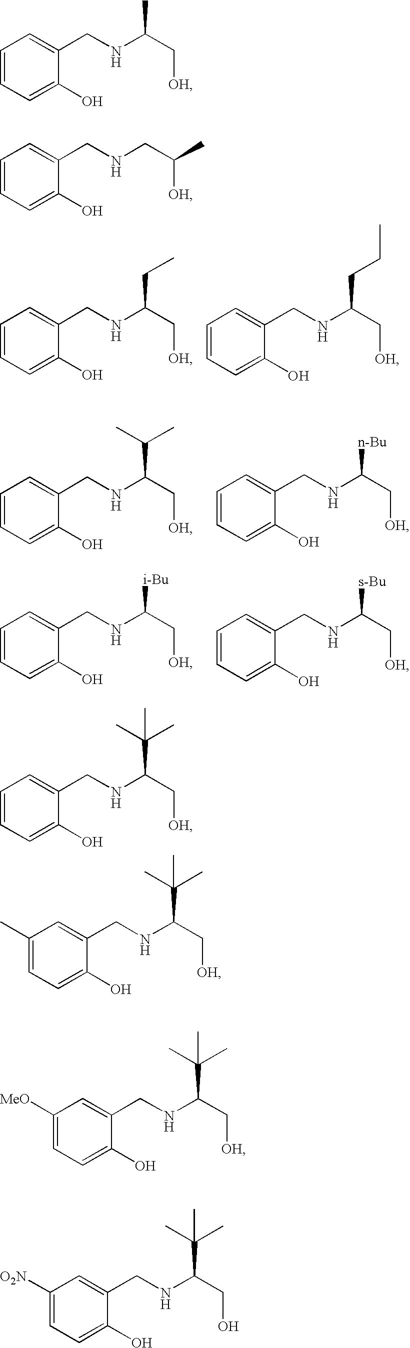

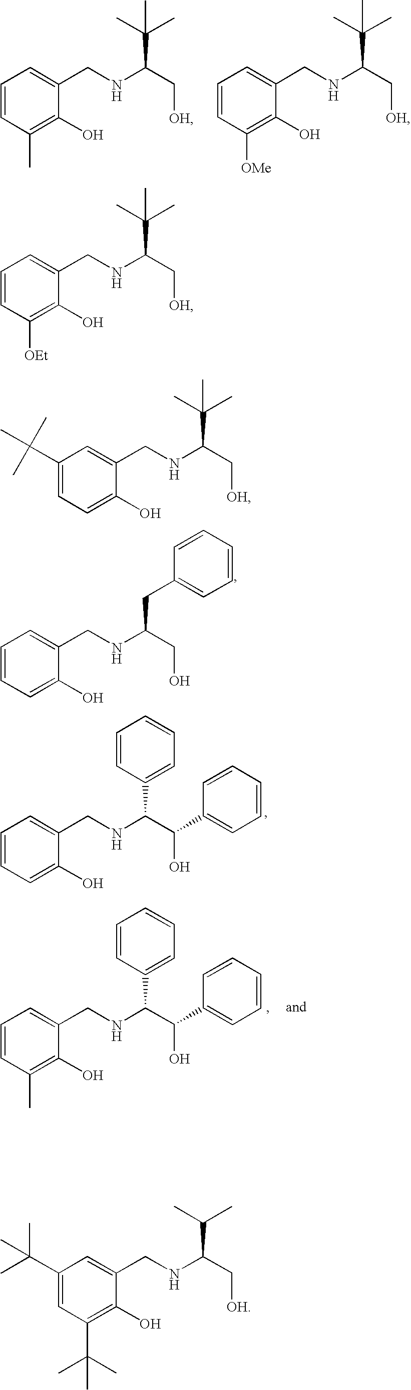

The titanium catalyst of the present invention can be produced by bringing a reaction mixture obtained by contacting water and a titanium alkoxide into contact with an optically active ligand represented by the general formula (a), as described above. The preparation of the titanium catalyst may further comprise use of a solvent, such as an organic solvent. For example, the reaction mixture may be obtained by combining the titanium alkoxide, in a mixture of water and an organic solvent, with the optically active ligand. In some cases, the organic solvent may comprise an amount of water. The molar ratio of the titanium alkoxide, water, and the optically active ligand represented by general formula (a) can be in the range of 1.0:0.1:0.1 to 1.0:2.0:3.0. Any molar ratio within this range may be suitable for use in the present invention.

In some embodiments, the optically active titanium catalyst is prepared by first reacting a titanium alkoxide (e.g., a titanium tetraalkoxide) compound with a hydrolyzing agent in an organic solvent to form a partially hydrolyzed titanium alkoxide species. In some cases, the hydrolyzing agent is water, or a water source. The water source (herein referred to as “water”) may be, for example, an inorganic hydrate (e.g., an inorganic salt comprising water molecules). Examples of inorganic hydrates include, but are not limited to, Na₂B₄O₇.10H₂O, Na₂SO₄.10H₂O, Na₃PO₄.12H₂O, MgSO₄.7H₂O, CuSO₄.5H₂O, FeSO₄.7H₂O, AlNa(SO₄)₂.12H₂O, AlK(SO₄)₂.12H₂O, and the like. When a moisture-absorbed molecular sieve is used, commercial products such as molecular sieves 3A, 4A, and the like exposed to outdoor air may be used, and any of a powder molecular sieve and a pellet molecular sieve can be used. In addition, undehydrated silica gel or zeolite may also be used as a water source. Further, when an inorganic hydrate or a molecular sieve is used, it can easily be removed from the reaction mixture by filtering before reaction with a ligand (e.g., an optically active ligand). At that time, water may be contained in an amount of from about 0.1 to 2.0 moles, or from about 0.2 to 1.5 moles, or even about 1 mole, based on 1 mole of the titanium alkoxide compound. Water in that amount is added and stirred. At that time, the titanium alkoxide compound may be dissolved in a solvent in advance and water may be diluted in a solvent, prior to addition. Water can also be directly added by a method comprising adding water in mist form, a method comprising using a reaction vessel equipped with a high efficiency stirrer or the like.

Examples of organic solvents suitable for use in the invention include halogenated hydrocarbon solvents such as dichloromethane, chloroform, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane and the like. In some embodiments, halogenated solvents or aromatic hydrocarbon solvents are used. The total amount of the solvent used when water is added may be from about 1 to 500 mL, or from about 10 to 50 mL, based on 1 millimole of the titanium alkoxide compound. It should be noted that use of the partially hydrolyzed titanium precursor can lead to an overall increased conversion rate and enantioselectivity in the asymmetric cyanation of imines.

The temperature at which the titanium alkoxide is reacted with water may be any temperature which does not freeze the solvent. For example, the reaction may be carried out at about room temperature, for example, from 15 to 30° C. The reaction may also be carried out at higher temperatures (e.g., by heating) depending on the boiling point of the solvent in use. The time required for the reaction is different depending on general conditions such as the amount of water to be added, the reaction temperature, and the like. In some embodiments, the time required for stirring is about 30 minutes to achieve formation of the titanium catalyst.

Next, the optically active ligand can be added and stirred. The optically active ligand may be added in an amount based on the titanium alkoxide compound with water such that a molar ratio of titanium to the optically active ligand may be from about 0.5:1 to 1:4, or any molar ratio within that range. In some embodiments, the molar ratio of Ti:optically active ligand may be about 1:1 to 1:3. In some embodiments, the molar ration of ratio of Ti:optically active ligand is 1:1.

In some embodiments, the optically active ligand may be dissolved in a solvent or may be added as it is without being dissolved. When a solvent is used, the solvent can be the same solvent as or different from the solvent used in the above step of adding water. When a solvent is newly added, the amount thereof may be from about 1 to about 5.000 mL, or from about 1 to about 500 mL, based on 1 millimole of the titanium atom. At this time, the reaction temperature is not particularly limited, but the compound can be usually produced by stirring at about room temperature, for example, from 15 to 30° C. for about 5 minutes to about 1 hour, or from about 30 minutes to about 1 hour.

In some cases, the production of the titanium compound of the present invention may advantageously be carried out under ambient conditions. However, it should also be understood that the production of the titanium compound of the present invention may be carried out under a dry and/or inert gas atmosphere or without strictly following dry and inert conditions. Examples of the inert gas include nitrogen, argon, helium and the like.

Subsequent to stirring of the reaction mixture the titanium compound (e.g., titanium catalyst) of the present invention can be obtained.

As described herein, one or more solvents may be used in the preparation of the optically active titanium catalyst. In some cases, use of a solvent may facilitate formation of the titanium compound. The solvent may be selected to dissolve any one of the titanium alkoxide, optically active ligand, other component, or combinations thereof to facilitate formation of the catalyst. Examples of the solvent include halogenated hydrocarbon solvents such as dichloromethane, chloroform and the like; halogenated aromatic hydrocarbon solvents such as chlorobenzene, o-dichlorobenzene, fluorobenzene, trifluoromethylbenzene and the like; aromatic hydrocarbon solvents such as toluene, xylene and the like; ester solvents such as ethyl acetate and the like; and ether solvents such as tetrahydrofuran, dioxane, diethyl ether, dimethoxyethane and the like. In some embodiments, halogenated hydrocarbon solvents or aromatic hydrocarbon solvents may be used. In some embodiments, mixtures of the above solvents may also be used.

The total amount of the solvent used in the preparation of the optically active titanium catalyst may be from about 1 to about 5.000 mL or from about 10 to about 500 mL, based on 1 millimole of the titanium atom in the titanium alkoxide compound. The reaction temperature at this time is not particularly limited, but the reaction may typically be performed from about 15 to 30° C. The reaction time required for preparing the titanium catalyst may be in the range of about 5 minutes to 1 hour, or about 30 minutes to about 1 hour. In some cases, the reaction time required for preparing the titanium catalyst is 30 minutes.

One advantageous feature of the present invention is that the titanium compound produced as above can be used in asymmetric catalytic reactions without need for further purification. That is, the titanium compound may be prepared and used directly in a subsequent asymmetric reaction, optionally in the same reaction vessel in which the titanium compound was prepared. This may eliminate the need for purification steps or additional synthetic steps, and reduces the production of waste materials, such as solvents and impurities.

Some embodiments of the invention provide processes for producing optically active alpha-aminonitriles. In methods of the invention, an imine substrate may be used as a starting material. The method may comprise reacting the imine substrate with a cyanating agent in the presence of a titanium catalyst as described herein, optionally in the presence of solvents, additives, and the like. In some cases, the imine is an unsymmetrical imine, that is, the imine has at least two different substituents on the carbon of the C═N bond. In some cases, the imine is a prochiral compound and can be suitably selected to correspond to the desired optically active alpha-aminonitrile product upon asymmetric cyanation of the imine.

In some cases, processes of the invention may comprise use of an imine represented by the general formula (c),

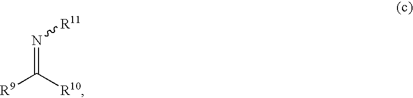

wherein R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰; R⁹ and R¹⁰ may be linked together to form a ring, and the ring may have a substituent; R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl or sulfoxy group, each of which may have a substituent; R¹¹ may be linked either to R⁹ or R¹⁰ to form a ring through a carbon chain, and the ring may have substituents.

In some embodiments, R⁹ is an alkyl group or an aryl group, R¹⁰ is a hydrogen atom, and R¹¹ is an alkyl group or an aryl group. In some embodiments, R⁹ is a hydrogen atom, and R¹⁰ and R¹¹ are independently an alkyl group or an aryl group.

Examples of R⁹ or R¹⁰ include, but not limited to, phenyl, 2-chlorophenyl, 2-bromophenyl, 2-fluorophenyl, 2-methylphenyl, 2-methoxyphenyl, 4-chlorophenyl, 4-bromophenyl, 4-fluorophenyl, 4-methylphenyl, 4-methoxyphenyl, 4-trifluoromethylphenyl, 4-nitrophenyl, furanyl, pyridyl, cinnamyl, 2-phenylethyl, methyl, ethyl, n-propyl, i-propyl, n-butyl, i-butyl, s-butyl, t-butyl, pentyl, hexyl, and the like.

Examples of R¹¹ include, benzyl, benzhydryl, 9-fluorenyl, 2-hydroxyphenyl, 4-methoxyphenyl, allyl, t-butoxycarbonyl, benzyloxycarbonyl, diphenylphosphinoyl, p-tolylsulfinyl, p-toluenesulfonyl, mesitylenesulfonyl and the like. R¹¹ may also be part of a ring as in 3,4-dihydroisoquinoline, and the like.

The imine substrates described herein may be synthesized by methods known in the art, for example, by condensation of an aldehyde or ketone with an amine to produce the corresponding imine substrate.

The process involves the use of a cyanating agent as a source of cyanide ion in the asymmetric cyanation reaction. Examples of cyanating agents suitable for use in the present invention include, but are not limited to, hydrogen cyanide, trialkylsilyl cyanide, acetone cyanohydrin, cyanoformate ester, potassium cyanide-acetic acid, potassium cyanide-acetic anhydride, tributyltin cyanide, and the like. In some embodiments, the cyanating agent is trialkylsilyl cyanide. In some cases, the cyanating agent is used in the reaction in an amount from 1 to 3 moles, or, in some cases, from 1.05 to 2.5 moles, or from 1.5 to 2.5 moles, based on 1 mole of the imine substrate. In some embodiments, 1.5 equivalents of cyanating agent may be used, based on the imine substrate.

As described herein, one or more solvents may be used in the asymmetric cyanation of imines. Examples of the solvent include halogenated hydrocarbon solvents such as dichloromethane, chloroform and the like; halogenated aromatic hydrocarbon solvents such as chlorobenzene, o-dichlorobenzene, fluorobenzene, trifluoromethylbenzene and the like; aromatic hydrocarbon solvents such as toluene, xylene and the like; ester solvents such as ethyl acetate and the like; ester solvents such as ethyl acetate and the like; and ether solvents such as tetrahydrofuran, dioxane, diethyl ether, dimethoxyethane and the like. In some embodiments, the solvent is a halogenated hydrocarbon solvent or aromatic hydrocarbon solvent. The solvents can be used alone or in combination as a mixture of solvents. In some embodiments, the total amount of solvent used may be about 0.1-5 mL, or, in some cases, 0.2-1 mL, based on 1 mmol of imine as a substrate.

The reactions described herein may be carried out by preparing the optically active titanium catalysts using methods as described herein, and then adding the imine substrate and cyanating agent to the titanium catalyst. The resulting mixture may be stirred at any reaction temperature, for example, from −78-80° C., or greater, for about 15 minutes to 6 hours, to produce the optically active alpha-aminonitrile product. In some embodiments, the mixture is stirred at a reaction temperature from about 0-30° C.

In some embodiments, methods of the present invention comprise use of the titanium catalyst in asymmetric reactions in an amount from 0.01 to 30 mole %, from 0.25 to 10 mole %, 2.5 to 10 mole %, or, 2.5 to 5.0 mole %, based on 1 mole of imine in terms of the titanium atom.

The temperature at which the asymmetric cyanation reaction occurs may be any temperature which does not freeze the components of the reactions, including the catalyst, imine substrate, cyanating agent, or other optional components including solvents and additives. In some cases, the reaction may be carried out at about room temperature, for example, from 15 to 30° C. The reaction may also be carried out at higher temperatures (e.g., by heating) depending on the boiling point of the solvent in use. The time required for the reaction is different depending on general conditions such as the reaction temperature, and the like. In some cases, the reaction time is six hours or less, 4 hours or less, two hours or less, 1 hour or less, 45 minutes or less, 30 minutes or less, or in some cases, 15 minutes or less. In some embodiments, the time required for stirring is about 15-60 minutes to achieve formation of the optically active alpha-aminonitrile product in high yield and with high enantioselectivity.

In some cases, the asymmetric cyanation reaction may advantageously be carried out under ambient conditions. However, it should also be understood that the production of the titanium compound of the present invention may be carried out under a dry and/or inert gas atmosphere or without strictly following dry and inert conditions. Examples of the inert gas include nitrogen, argon, helium and the like. Subsequent to stirring of the reaction mixture, the optically active alpha-aminonitrile product can be obtained.

In some embodiments, an additive may also be used in the asymmetric cyanation of imines. For example, the additive may be added to the mixture comprising the titanium catalyst, the imine substrate, the cyanating agent, and/or solvent. The additive may be added at any time during the reaction, i.e., during preparation of the titanium catalyst and/or during cyanation of the imine substrate. The additive may be, for example, a species comprising at least one hydroxyl group (e.g., water, alcohols, diols, polyols, etc.). In some embodiments, the additive is water. In some embodiments, the additive is an alcohol. Examples of the alcohols suitable for use as an additive include an aliphatic alcohol and an aromatic alcohol, each of which may have a substituent, and/or combinations thereof. In some cases the alcohol is an alkyl alcohol, including linear, branched or cyclic alkyl alcohols having 10 carbon atoms or less. Some examples of alkyl alcohols include methanol, ethanol, n-propanol, isopropanol, n-butanol, sec-butanol, tert-butanol, cyclopentyl alcohol, cyclohexyl alcohol and the like. The alkyl alcohol may have one or more substituents, including a halogen atom such as a fluorine atom, a chlorine atom, a bromine atom, an iodine atom and the like. Examples of alkyl alcohols having a halogen atom include halogenated alkyl alcohols having 10 carbon atoms or less, such as chloromethanol, 2-chloroethanol, trifluoromethanol, 2,2,2-trifluoroethanol, perfluoroethanol, perfluorohexyl alcohol and the like.

In some cases, the alcohol may be an aromatic alcohol, including aryl alcohols having 6 to 20 carbon atoms. Some examples of aryl alcohols include phenol, naphthol and the like. The aryl alcohol may have one or more substituents on the aryl group, including a halogen atom such as a fluorine atom, a chlorine atom, a bromine atom, an iodine atom and the like, or an alkyl group having 20 carbon atoms or less. Examples of aryl alcohols having a halogen atom include halogenated aryl alcohols having 6 to 20 carbon atoms such as pentafluorophenol and the like. Examples of aryl alcohols having an alkyl group include dimethylphenol, trimethylphenol, isopropylphenol, diisopropylphenol, tert-butylphenol, di-tert-butylphenol and the like.

In some cases, the additive may comprise more than one hydroxyl group. For example, the additive may be a diol or polyol.

In some cases, the additive may be added in an amount that is 0.25 equivalents, 0.5 equivalents, 1.0 equivalent, 1.5 equivalents, 2.0 equivalents, or greater, based on the amount of the imine substrate.

In some cases, the additive may be added as a neat reagent or added as a solution in a solvent.

In some cases, the additive may be one or more compounds.

In some embodiments, when water is used as an additive in the asymmetric cyanation reaction, the titanium catalyst may be prepared by using an inorganic hydrate as the hydrolyzing agent. In some embodiments, when alcohol is used as an additive in the asymmetric cyanation reaction, the titanium catalyst may be prepared using residual water in toluene (e.g., 200-400 ppm) as the hydrolyzing agent.

In one set of embodiments, high catalytic activity and enantioselectivity may be observed when water or an alcohol such as n-butanol is used as an additive in the asymmetric cyanation of imines, using the titanium catalysts described herein. In some embodiments, substantially complete conversion of the imine substrate to the desired optically active alpha-aminonitrile can be achieved in 15 minutes with the addition of 0.5 equivalent of water or 1.0 equivalent of n-butanol. In some cases, enantioselectivities of at least 80% ee, at least 85% ee, at least 90% ee, at least 95% ee, at least 98% ee, can be observed. In a particular embodiment, the asymmetric cyanation of imines may be performed with 2.5 to 5 mole % of a titanium catalyst as described herein, at room temperature, to produce a product in >99% yield and having up to 98% ee, in 15 minutes.

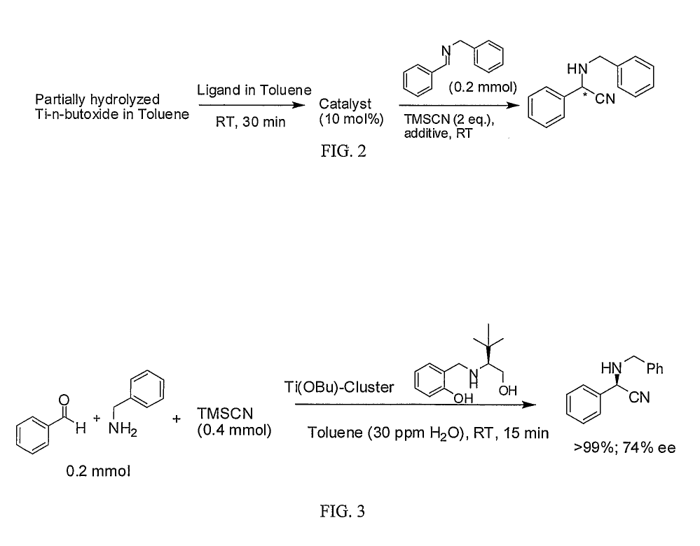

In some cases, methods of the invention may involve a “one pot” synthesis. That is, the present invention may involve an (at least) three component, one-pot synthesis of alpha-aminonitriles. The term “one-pot” reaction is known in the art and refers to a chemical reaction which can produce a product in one step which may otherwise have required a multiple-step synthesis, and/or a chemical reaction comprising a series of steps that may be performed in a single reaction vessel. One-pot procedures may eliminate the need for isolation (e.g., purification) of intermediates and additional synthetic steps while reducing the production of waste materials (e.g., solvents, impurities). Additionally, the time and cost required to synthesize such compounds may be reduced. In one embodiment, the “one pot” synthesis may comprise the simultaneous addition of at least some components of the reaction to a single reaction chamber. In one embodiment, the “one pot” synthesis may comprise sequential addition of various reagents to a single reaction chamber. In some embodiments, the asymmetric cyanation of imines may be performed as a one-pot reaction, wherein the imine substrate is formed in situ, using an aldehyde and an amine are used as substrates. For example, in some cases, the imine may be generated in situ by reacting a carbonyl compound in presence of a primary amine. FIG. 3 shows a “one-pot” synthesis of an alpha-aminonitrile, according to one embodiment of the invention.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

The present invention is now more specifically illustrated below with reference to Examples. However, the present invention is not restricted to these Examples.

### Example 1

The following example describes a general procedure for the preparation of titanium compounds (e.g., catalysts), as described herein. Ti(On-Bu)₄ (0.5 mmol) and 0.1 equiv. of Na₂B₄O₇.10H₂O were placed in a reaction vial in a glovebox, and 3 mL of dry toluene (10-30 ppm of water) were added. The solution was stirred under nitrogen atmosphere for 18 h at room temperature. The solution was then filtered and dry toluene (10-30 ppm water) was added to form a 10 mL solution, which was stirred further for 24-72 h to obtain a 0.05 M toluene solution of partially hydrolyzed Ti(On-Bu)₄ pre-catalyst.

Alternatively, the partially hydrolyzed Ti-alkoxide pre-catalyst was prepared using toluene having 200-400 ppm water. Ti(On-Bu)₄ (0.5 mmol) was placed in a reaction vial in a glovebox, and 10 mL of toluene having 200-400 ppm water was added. The solution was stirred for 18-72 h at room temperature to obtain a 0.05 M toluene solution of partially hydrolyzed Ti(On-Bu)₄ pre-catalyst.

Both methods can also be carried out without maintaining strictly inert conditions such as adding toluene outside the glovebox and stirring for the desired time.

Finally, the chiral titanium catalyst was prepared in situ by stirring the 0.05 M toluene solution of partially hydrolyzed Ti(On-Bu)₄ (200 microliters) with the optically active ligand shown in Table 1 in 500 microliters of toluene for 30 minutes.

### Example 2

The following example describes a general procedure for the use of titanium compounds in the asymmetric cyanation of imines, as described herein. The chiral titanium catalyst, prepared according to the methods described in Example 1, was used in the asymmetric cyanation reaction shown in FIG. 2. The chiral titanium catalyst (10 mol % based on the imine substrate) was placed in a flask, and N-benzylbenzylidineamine (0.2 mmol) and trimethylsilyl cyanide (2 equivalents based on the imine substrate) were added. The resulting material was stirred at room temperature for 20 hours, and NMR and HPLC analysis were carried out to determine the yield and enantiomeric excess (ee) of the product. The results are shown in Table 1.

### Example 3

The asymmetric cyanation reaction was carried out in the same manner as in Example 2 except that the optically active ligand as shown in Table 1 was used. The results are shown in Table 1.

### Example 4

The asymmetric cyanation reaction was carried out in the same manner as in Example 2 except that the optically active ligand as shown in Table 1 was used. The results are shown in Table 1.

### Example 5

The asymmetric cyanation reaction was carried out in the same manner as in Example 2 except that the optically active ligand as shown in Table 1 was used and the reaction was stirred at room temperature for 47 hours. The results are shown in Table 1.

### Example 6

The asymmetric cyanation reaction was carried out in the same manner as in Example 2 except that the optically active ligand as shown in Table 1 was used. The results are shown in Table 1.

TABLE 1
Screening of ligands for asymmetric cyanation of imines.

| Exam- | | | conv. | |
|---|---|---|---|---|
| ple | Ligand | Time, h | % | ee, % |
| 1 | 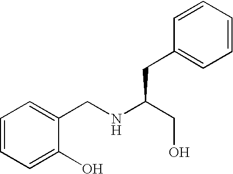 | 20 | 49 | 47.1 |
| 3 | 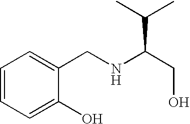 | 20 | 55 | 64.1 |
| 4 | 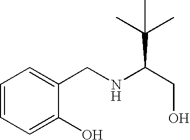 | 20 | 59 | 76.8 |
| 5 | 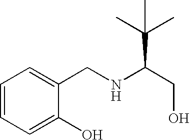 | 47 | 67 | 69.6 |
| 6 | 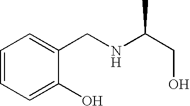 | 20 | 60 | 35.8 |

### Example 7

In the following example, the asymmetric cyanation reaction was carried out using an alcohol as an additive. The chiral titanium catalyst was prepared in situ by stirring the required amount of partially hydrolyzed Ti(On-Bu)₄ in toluene for 30 min together with the optically active ligand shown in Example 4, with water content and mmol ratio of Ti:water during partial hydrolysis partial hydrolysis as indicated in Table 2.

The chiral titanium catalyst was then used directly in the asymmetric cyanation of imines, according to the following general procedure. The chiral titanium catalyst (10 mol % based on the imine substrate) was placed in a flask, and N-benzylbenzylidine-amine (0.2 mmol), trimethylsilyl cyanide (2 equivalents relative to the imine substrate), and butanol (1.0 equivalent based on the imine substrate) as an additive, were added in order. The resulting material was stirred at room temperature for 2 hours, and NMR and HPLC analysis were carried out to determine the yield and enantiomeric excess (ee) of the product. The results are shown in Table 2.

### Example 8

The asymmetric cyanation reaction was carried out in the same manner as in Example 7 except that the reaction was stirred at room temperature for 4 hours. The results are shown in Table 2.

### Example 9

The asymmetric cyanation reaction was carried out in the same manner as in Example 7 except that 1.5 equivalents of butanol were used based on the imine substrate, and the reaction was stirred at room temperature for 1 hour. The results are shown in Table 2.

### Example 10

The asymmetric cyanation reaction was carried out in the same manner as in Example 7 except that 0.5 equivalents of butanol were used based on the imine substrate. The results are shown in Table 2.

### Example 11

The asymmetric cyanation reaction was carried out in the same manner as in Example 7 except that residual water was used as the hydrolyzing agent, with a water content and mmol ratio of Ti:water, during partial hydrolysis, as indicated in Table 2. The results are shown in Table 2.

### Example 12

The asymmetric cyanation reaction was carried out in the same manner as in Example 7 except that residual water was used as the hydrolyzing agent, with a water content and mmol ratio of Ti:water, during partial hydrolysis, as indicated in Table 2. The reaction was stirred at room temperature for 15 minutes. The results are shown in Table 2.

### Example 13

The asymmetric cyanation reaction was carried out in the same manner as in Example 7 except that water (0.5 equivalents based on the imine substrate) was used as the additive, and the reaction was stirred at room temperature for 15 min. The results are shown in Table 2.

### Example 14

The asymmetric cyanation reaction was carried out in the same manner as in Example 7 except that water (0.5 equivalents based on the imine substrate) was used as the additive, and the reaction was stirred at room temperature for 30 min. The results are shown in Table 2.

### Example 15

The asymmetric cyanation reaction was carried out in the same manner as in Example 7 except that water (0.5 equivalents based on the imine substrate) was used as the additive. The results are shown in Table 2.

### Example 16

The asymmetric cyanation reaction was carried out in the same manner as in Example 7 except that water (1.0 equivalent based on the imine substrate) was used as the additive. The results are shown in Table 2.

### Example 17

The asymmetric cyanation reaction was carried out in the same manner as in Example 7 except that water (1.5 equivalents based on the imine substrate) was used as the additive. The results are shown in Table 2.

### Example 18

The asymmetric cyanation reaction was carried out in the same manner as in Example 7 except that water (0.5 equivalents based on the imine substrate) was used as the additive. The results are shown in Table 2.

### Example 19

The asymmetric cyanation reaction was carried out in the same manner as in Example 7 except that water (0.25 equivalents based on the imine substrate) was used as the additive, and the reaction was stirred at room temperature for 15 min. The results are shown in Table 2.

### Example 20

The asymmetric cyanation reaction was carried out in the same manner as in Example 7 except that water (0.25 equivalents based on the imine substrate) was used as the additive, and the reaction was stirred at room temperature for 1 hour. The results are shown in Table 2.

### Example 21

The asymmetric cyanation reaction was carried out in the same manner as in Example 7 except that residual water was used as the hydrolyzing agent, with a water content and mmol ratio of Ti:water, during partial hydrolysis, as indicated in Table 2. Water (0.5 equivalents based on the imine substrate) was used as the additive, and the reaction was stirred at room temperature for 15 min. The results are shown in Table 2.

### Example 22

The asymmetric cyanation reaction was carried out in the same manner as in Example 7 except that residual water was used as the hydrolyzing agent, with a water content and mmol ratio of Ti:water, during partial hydrolysis, as indicated in Table 2. Water (0.5 equivalents based on the imine substrate) was used as the additive, and the reaction was stirred at room temperature for 45 min. The results are shown in Table 2.

### Example 23

The asymmetric cyanation reaction was carried out in the same manner as in Example 7 except that residual water was used as the hydrolyzing agent, with a water content and mmol ratio of Ti:water, during partial hydrolysis, as indicated in Table 2. Water (0.25 equivalents based on the imine substrate) was used as the additive, and the reaction was stirred at room temperature for 15 min. The results are shown in Table 2.

### Example 24

The asymmetric cyanation reaction was carried out in the same manner as in Example 7 except that residual water was used as the hydrolyzing agent, with a water content and mmol ratio of Ti:water, during partial hydrolysis, as indicated in Table 2. Water (0.25 equivalents based on the imine substrate) was used as the additive, and the reaction was stirred at room temperature for 30 min. The results are shown in Table 2.

### Example 25

The asymmetric cyanation reaction was carried out in the same manner as in Example 7 except that residual water was used as the hydrolyzing agent, with a water content and mmol ratio of Ti:water, during partial hydrolysis, as indicated in Table 2. Water (0.25 equivalents based on the imine substrate) was used as the additive, and the reaction was stirred at room temperature for 1 hour. The results are shown in Table 2.

TABLE 2
Effect of additives with different partially hydrolyzed Ti(OnBu)₄.

| | | Water | | | | | |
|---|---|---|---|---|---|---|---|
| | Hydrolyzing | content | Ti:H₂O, mmol, | | | | |
| | Agent for | of | during partial | Additive, | | | |
| Example | Ti(OnBu)₄ | Toluene | hydrolysis | equivalent | Time | Conv. % | ee % |
| 7 | Inorganic | 30 ppm | 0.5:0.516 | Butanol, | 2 h | 90 | 84.7 |
| | hydrate | | | 1.0 | | | |
| 8 | Inorganic | 30 ppm | 0.5:0.516 | Butanol, | 4 h | >99 | 82.5 |
| | hydrate | | | 1.0 | | | |
| 9 | Inorganic | 30 ppm | 0.5:0.516 | Butanol, | 1 h | >99 | 80.3 |
| | hydrate | | | 1.5 | | | |
| 10 | Inorganic | 30 ppm | 0.5:0.516 | Butanol, | 2 h | 90 | 83.3 |
| | hydrate | | | 0.5 | | | |
| 11 | Residual | 200 ppm | 0.5:0.111 | Butanol, | 2 h | >99 | 87.0 |
| | water | | | 1.0 | | | |
| 12 | Residual | 380 ppm | 0.5:0.211 | Butanol, | 15 min | >99 | 85.5 |
| | water | | | 1.0 | | | |
| 13 | Inorganic | 30 ppm | 0.5:0.516 | Water, 0.5 | 15 min | 94 | 85.6 |
| | hydrate | | | | | | |
| 14 | Inorganic | 30 ppm | 0.5:0.516 | Water, 0.5 | 30 min | >98 | 85.0 |
| | hydrate | | | | | | |
| 15 | Inorganic | 30 ppm | 0.5:0.516 | Water, 0.5 | 2 h | >99 | 85.0 |
| | hydrate | | | | | | |
| 16 | Inorganic | 30 ppm | 0.5:0.516 | Water, 1.0 | 2 h | >99 | 77.0 |
| | hydrate | | | | | | |
| 17 | Inorganic | 30 ppm | 0.5:0.516 | Water, 1.5 | 2 h | >99 | 61.0 |
| | hydrate | | | | | | |
| 18 | Inorganic | 30 ppm | 0.5:0.516 | Water, 0.5 | 2 h | >99 | 85.0 |
| | hydrate | | | | | | |
| 19 | Inorganic | 30 ppm | 0.5:0.516 | Water, | 15 min | 85 | 84.9 |
| | hydrate | | | 0.25 | | | |
| 20 | Inorganic | 30 ppm | 0.5:0.516 | Water, | 1 h | 89 | 85.0 |
| | hydrate | | | 0.25 | | | |
| 21 | Residual | 200 ppm | 0.5:0.111 | Water, 0.5 | 15 min | 83 | 84.0 |
| | water | | | | | | |
| 22 | Residual | 200 ppm | 0.5:0.111 | Water, 0.5 | 45 min | 89 | 83.0 |
| | water | | | | | | |
| 23 | Residual | 200 ppm | 0.5:0.111 | Water, | 15 min | 78 | 84.6 |
| | water | | | 0.25 | | | |
| 24 | Residual | 200 ppm | 0.5:0.111 | Water, | 30 min | 84 | 85.6 |
| | water | | | 0.25 | | | |
| 25 | Residual | 200 ppm | 0.5:0.111 | Water, | 1 h | 86 | 86.0 |
| | water | | | 0.25 | | | |

### Example 26

In the following example, the asymmetric cyanation reaction was carried out using an alcohol as an additive. The chiral titanium catalyst was prepared in situ by stirring the required amount of partially hydrolyzed Ti(On-Bu)₄ in toluene for 30 min together with the optically active ligand as shown in Example 4 and residual water (200 ppm) during partial hydrolysis.

The chiral titanium catalyst was then used directly in the asymmetric cyanation of imines, according to the following general procedure. The chiral titanium catalyst (10 mol % based on the imine substrate) was placed in a flask, and N-benzylbenzylidine-amine (0.2 mmol), trimethylsilyl cyanide (1.5 equivalents relative to the imine substrate), and butanol (1.0 equivalent based on the imine substrate) as an additive, were added in order. The reaction mixture was stirred at room temperature for 15 min, and NMR and HPLC analysis were carried out to determine the yield and enantiomeric excess (ee) of the product. The results are shown in Table 3.

### Example 27

The asymmetric cyanation reaction was carried out in the same manner as in Example 26 except that Ti(OEt)₄ was used to prepare the chiral titanium catalyst. The results are shown in Table 3.

### Example 28

The asymmetric cyanation reaction was carried out in the same manner as in Example 26 except that Ti(OiPr)₄ was used to prepare the chiral titanium catalyst. The results are shown in Table 3.

TABLE 3
Effect of partially hydrolyzed Ti alkoxides prepared from various Ti
alkoxide monomers.

| Example | Ti(OR)₄ | TMSCN, equiv. | Time | Conv. % | ee, % |
|---|---|---|---|---|---|
| 26 | Ti(OnBu)₄ | 1.5 | 15 min | >99 | 87.0 |
| 27 | Ti(OEt)₄ | 1.5 | 15 min | >99 | 87.0 |
| 28 | Ti(OiPr)₄ | 1.5 | 15 min | >99 | 87.0 |

### Example 29

In the following example, the asymmetric cyanation reaction was carried out without strictly following inert conditions. The chiral titanium catalyst was prepared in situ by stirring the required amount of partially hydrolyzed Ti(On-Bu)₄ in toluene for 30 min together with the optically active ligand as shown in Example 4 and residual water (200 ppm) during partial hydrolysis.

The chiral titanium catalyst was then used directly in the asymmetric cyanation of imines, according to the following general procedure. The chiral titanium catalyst (10 mol % based on the imine substrate) was placed in a flask, and N-benzylbenzylidine-amine (0.2 mmol), trimethylsilyl cyanide (2.0 equivalents relative to the imine substrate), and butanol as an additive (1.0 equivalent based on the imine substrate), were added in order. The resulting material was stirred at room temperature for 15 min, and NMR and HPLC analysis were carried out to determine the yield and enantiomeric excess (ee) of the product. The results are shown in Table 4.

### Example 30

The asymmetric cyanation reaction was carried out in the same manner as in Example 29, except that 5 mol % chiral titanium catalyst was used based on the imine substrate. The results are shown in Table 4.

### Example 31

The asymmetric cyanation reaction was carried out in the same manner as in Example 29, except that 2.5 mol % chiral titanium catalyst was used based on the imine substrate. The results are shown in Table 4.

### Example 32

The asymmetric cyanation reaction was carried out in the same manner as in Example 29, except that 2.5 mol % chiral titanium catalyst was used based on the imine substrate and the reaction was stirred at room temperature for 30 min. The results are shown in Table 4.

### Example 33

The asymmetric cyanation reaction was carried out in the same manner as in Example 29, except that 1.0 mol % chiral titanium catalyst was used based on the imine substrate. The results are shown in Table 4.

### Example 34

The asymmetric cyanation reaction was carried out in the same manner as in Example 29, except that 1.0 mol % chiral titanium catalyst was used based on the imine substrate and the reaction was stirred at room temperature for 30 min. The results are shown in Table 4.

### Example 35

The asymmetric cyanation reaction was carried out in the same manner as in Example 29, except that 5.0 mol % chiral titanium catalyst and 1.5 equivalents of TMSCN were used, based on the imine substrate. The results are shown in Table 4.

### Example 36

The asymmetric cyanation reaction was carried out in the same manner as in Example 29, except that 5.0 mol % chiral titanium catalyst and 1.5 equivalents of TMSCN were used, based on the imine substrate, and the reaction was stirred at room temperature for 30 min. The results are shown in Table 4.

### Example 37

The asymmetric cyanation reaction was carried out in the same manner as in Example 29, except that 5.0 mol % chiral titanium catalyst and 1.0 equivalents of TMSCN were used, based on the imine substrate, and the reaction was stirred at room temperature for 30 min. The results are shown in Table 4.

### Example 38

The asymmetric cyanation reaction was carried out in the same manner as in Example 29, except that 5.0 mol % chiral titanium catalyst and 1.05 equivalents of TMSCN were used, based on the imine substrate, and the reaction was stirred at room temperature for 1 hour. The results are shown in Table 4.

TABLE 4
Effect of concentration of catalyst and TMSCN for the cyanation of N-
Benzylbenzylidineamine

| | Catalyst, | TMSCN, | | | |
|---|---|---|---|---|---|
| Example | mol % | equiv. | Time | Conv. % | ee, % |
| 29 | 10.0 | 2.0 | 15 min | >99 | 86.5 |
| 30 | 5.0 | 2.0 | 15 min | >99 | 86.0 |
| 31 | 2.5 | 2.0 | 15 min | 89 | 83.0 |
| 32 | 2.5 | 2.0 | 30 min | 95 | 84.0 |
| 33 | 1.0 | 2.0 | 15 min | 31 | 42.0 |
| 34 | 1.0 | 2.0 | 30 min | 42 | 42.0 |
| 35 | 5.0 | 1.5 | 15 min | 99 | 87.0 |
| 36 | 5.0 | 1.5 | 30 min | >99 | 86.5 |
| 37 | 5.0 | 1.0 | 30 min | 86 | 85.0 |
| 38 | 5.0 | 1.0 | 1 hr | 92 | 85.0 |

### Example 39

In the following example, the asymmetric cyanation reaction was carried out with according to the following general procedure. The chiral titanium catalyst was prepared in situ by stirring the required amount of partially hydrolyzed Ti(On-Bu)₄ in toluene with 200 ppm water for 30 min together with the optically active ligand as shown Table 5 (in toluene with 200 ppm water).

The chiral titanium catalyst was then used directly in the asymmetric cyanation of imines. The chiral titanium catalyst (5 mol % based on the imine substrate) was placed in a flask, and N-benzylbenzylidine-amine (0.2 mmol), trimethylsilyl cyanide (1.5 equivalents relative to the imine substrate), and butanol as an additive (1.0 equivalent based on the imine substrate), were added in order. The resulting material was stirred at room temperature for 15-60 min, and NMR and HPLC analysis were carried out to determine the yield and enantiomeric excess (ee) of the product. The results are shown in Table 5.

### Example 40

The asymmetric cyanation reaction was carried out in the same manner as in Example 39, except using the optically active ligand as indicated in Table 5. The results are shown in Table 5.

### Example 41

The asymmetric cyanation reaction was carried out in the same manner as in Example 39, except using the optically active ligand as indicated in Table 5. The results are shown in Table 5.

### Example 42

The asymmetric cyanation reaction was carried out in the same manner as in Example 39, except using the optically active ligand as indicated in Table 5. The results are shown in Table 5.

### Example 43

The asymmetric cyanation reaction was carried out in the same manner as in Example 39, except using the optically active ligand as indicated in Table 5. The results are shown in Table 5.

### Example 44

The asymmetric cyanation reaction was carried out in the same manner as in Example 39, except using the optically active ligand as indicated in Table 5. The results are shown in Table 5.

TABLE 5
Effect of chiral ligands for the cyanation of N-Benzylbenzylidineamine.

| Example | Optically Active Ligand | Conv. % | ee, % |
|---|---|---|---|
| 39 | 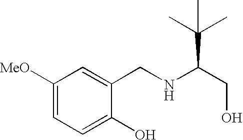 | >99 | 85 |
| 40 | 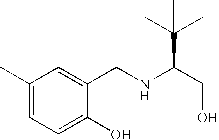 | >99 | 87 |
| 41 | 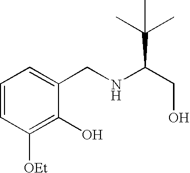 | >99 | 79 |
| 42 | 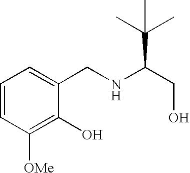 | >99 | 74 |
| 43 | 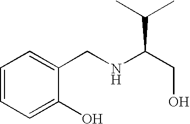 | >99 | 73 |
| 44 | 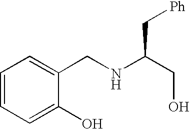 | >99 | 69 |

### Example 45

In the following example, the asymmetric cyanation reaction was carried out with according to the following general procedure. The chiral titanium catalyst was prepared in situ by stirring the required amount of partially hydrolyzed Ti(On-Bu)₄ in toluene having 200 ppm water for 30 min together with the optically active ligand as shown in Example 4 (in toluene with 200 ppm water).

The chiral titanium catalyst was then used directly in the asymmetric cyanation of imines. The chiral titanium catalyst (5 mol % based on the imine substrate) was placed in a flask, and the imine as indicated in Table 6 (0.2 mmol), trimethylsilyl cyanide (1.5 equivalents relative to the imine substrate), and butanol as an additive (1.0 equivalent based on the imine substrate), were added in order. The resulting material was stirred at room temperature for 15-60 min, and NMR and HPLC analysis were carried out to determine the yield and enantiomeric excess (ee) of the product. The results are shown in Table 6.

### Example 46

The asymmetric cyanation reaction was carried out in the same manner as in Example 45, except using the imine substrate as indicated in Table 6. The results are shown in Table 6.

### Example 47

The asymmetric cyanation reaction was carried out in the same manner as in Example 45, except using the imine substrate as indicated in Table 6. The results are shown in Table 6.

### Example 48

The asymmetric cyanation reaction was carried out in the same manner as in Example 45, except using the imine substrate as indicated in Table 6. The results are shown in Table 6.

### Example 49

The asymmetric cyanation reaction was carried out in the same manner as in Example 45, except using the imine substrate as indicated in Table 6. The results are shown in Table 6.

### Example 50

The asymmetric cyanation reaction was carried out in the same manner as in Example 45, except using the imine substrate as indicated in Table 6. The results are shown in Table 6.

### Example 51

The asymmetric cyanation reaction was carried out in the same manner as in Example 45, except using the imine substrate as indicated in Table 6. The results are shown in Table 6.

### Example 52

The asymmetric cyanation reaction was carried out in the same manner as in Example 45, except using the imine substrate as indicated in Table 6. The results are shown in Table 6.

### Example 53

The asymmetric cyanation reaction was carried out in the same manner as in Example 45, except using the imine substrate as indicated in Table 6. The results are shown in Table 6.

### Example 54

The asymmetric cyanation reaction was carried out in the same manner as in Example 45, except using the imine substrate as indicated in Table 6. The results are shown in Table 6.

### Example 55

The asymmetric cyanation reaction was carried out in the same manner as in Example 45, except using the imine substrate as indicated in Table 6. The results are shown in Table 6.

### Example 56

The asymmetric cyanation reaction was carried out in the same manner as in Example 45, except using the imine substrate as indicated in Table 6. The results are shown in Table 6.

### Example 57

The asymmetric cyanation reaction was carried out in the same manner as in Example 45, except using the imine substrate as indicated in Table 6. The results are shown in Table 6.

### Example 58

The asymmetric cyanation reaction was carried out in the same manner as in Example 45, except using the imine substrate as indicated in Table 6. The results are shown in Table 6.

### Example 59

The asymmetric cyanation reaction was carried out in the same manner as in Example 45, except using the imine substrate as indicated in Table 6. The results are shown in Table 6.

### Example 60

The asymmetric cyanation reaction was carried out in the same manner as in Example 45, except using the imine substrate as indicated in Table 6. The results are shown in Table 6.

### Example 61

The asymmetric cyanation reaction was carried out in the same manner as in Example 45, except using the imine substrate as indicated in Table 6. The results are shown in Table 6.

### Example 62

The asymmetric cyanation reaction was carried out in the same manner as in Example 45, except using the imine substrate as indicated in Table 6. The results are shown in Table 6.

### Example 63

The asymmetric cyanation reaction was carried out in the same manner as in Example 45, except using the imine substrate as indicated in Table 6. The results are shown in Table 6.

### Example 64

The asymmetric cyanation reaction was carried out in the same manner as in Example 45, except using the imine substrate as indicated in Table 6. The results are shown in Table 6.

### Example 65

The asymmetric cyanation reaction was carried out in the same manner as in Example 45, except using the imine substrate as indicated in Table 6. The results are shown in Table 6.

### Example 66

The asymmetric cyanation reaction was carried out in the same manner as in Example 45, except using the imine substrate as indicated in Table 6. The results are shown in Table 6.

### Example 67

The asymmetric cyanation reaction was carried out in the same manner as in Example 45, except using the imine substrate as indicated in Table 6. The results are shown in Table 6.

### Example 68

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 69

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 70

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 71

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 72

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 73

The asymmetric cyanation reaction was carried out in the same manner as in Example 45, except using the imine substrate as indicated in Table 6. After the reaction, trifluoroacetic anhydride was added to convert the aminonitrile to the trifluoroacetamide derivative for analysis. The results are shown in Table 6.

### Example 74

The asymmetric cyanation reaction was carried out in the same manner as in Example 45, except using the imine substrate as indicated in Table 6. The results are shown in Table 6.

### Example 75

The asymmetric cyanation reaction was carried out in the same manner as in Example 45, except using the imine substrate as indicated in Table 6. The results are shown in Table 6.

TABLE 6
Substrate scope for the asymmetric cyanation of imines.

| Example | Imine | Product | Conversion, % | ee, % |
|---|---|---|---|---|
| 45 | 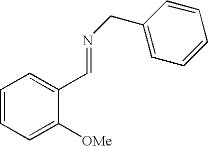 | 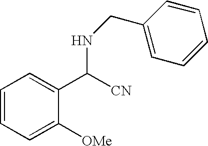 | >99 | 48 |
| 46 | 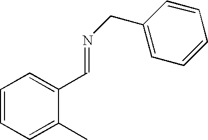 | 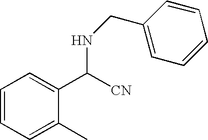 | >99 | 80 |
| 47 | 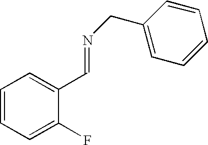 | 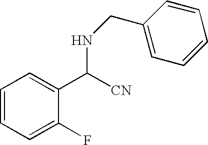 | >99 | 84 |
| 48 | 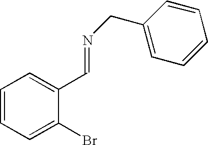 | 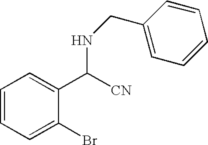 | >99 | 91 |
| 49 | 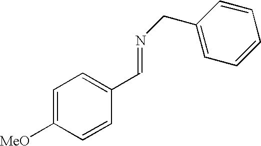 | 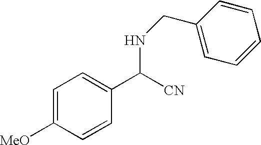 | >99 | 78 |
| 50 | 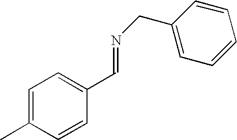 | 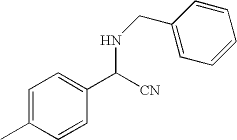 | >99 | 87 |
| 51 | 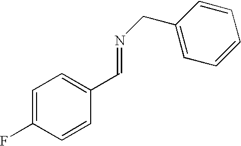 | 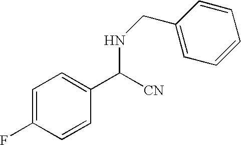 | >99 | 77 |
| 52 | 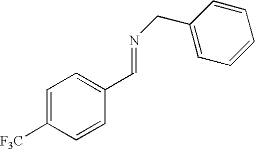 | 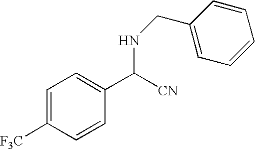 | >99 | 87 |
| 53 | 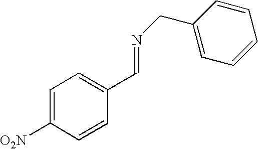 | 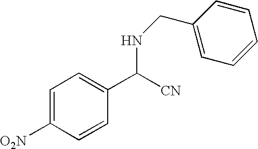 | >99 | 84 |
| 54 | 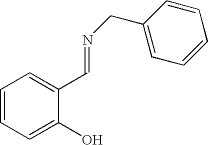 | 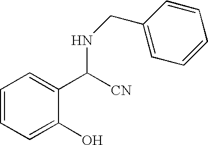 | >99 | 49 |
| 55 | 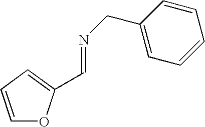 | 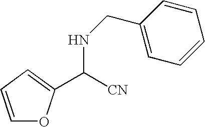 | >99 | 62 |
| 56 | 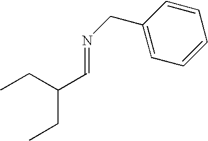 | 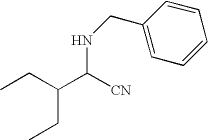 | >99 | 46 |
| 57 | 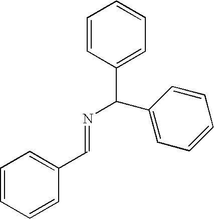 | 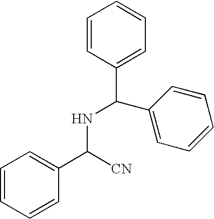 | >99 | 96 |
| 58 | 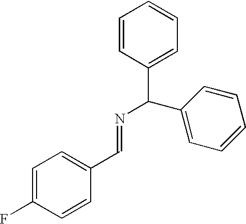 | 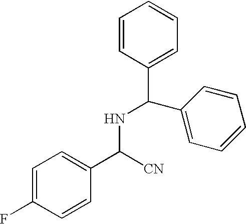 | 95 | 92 |
| 59 | 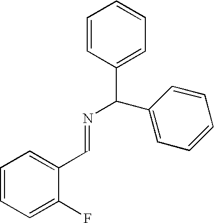 | 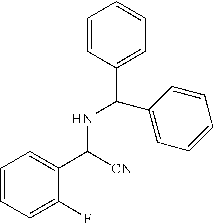 | 97 | 93 |
| 60 | 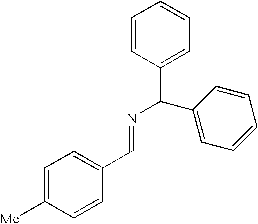 | 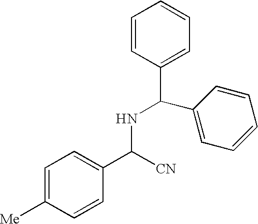 | >99 | 97 |
| 61 | 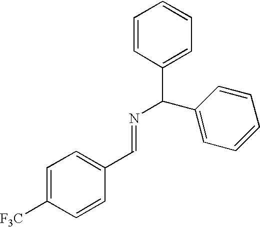 | 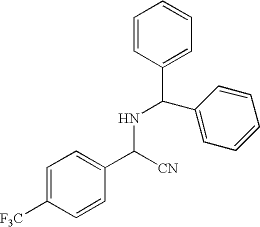 | >99 | 83 |
| 62 | 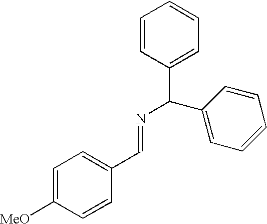 | 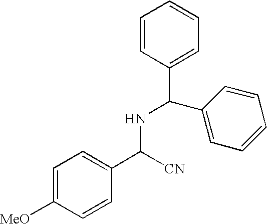 | >99 | 76 |
| 63 | 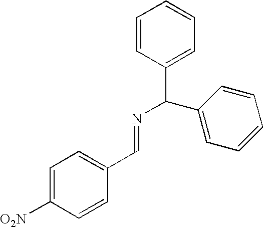 | 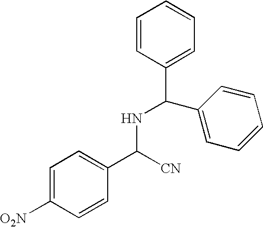 | 82 | 77 |
| 64 | 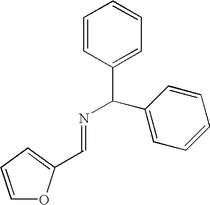 | 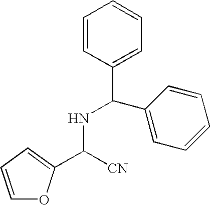 | >99 | 89 |
| 65 | 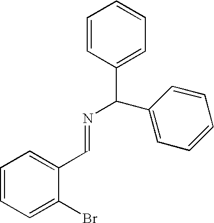 | 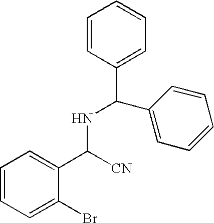 | >99 | 97 |
| 66 | 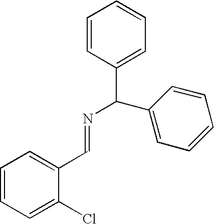 | 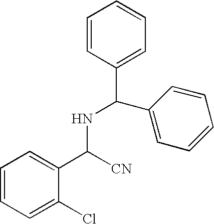 | >99 | 98 |
| 67 | 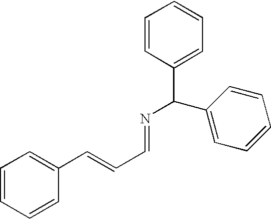 | 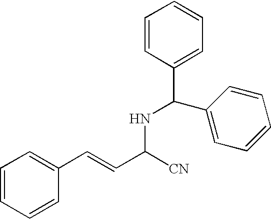 | >99 | 97 |
| 68 | 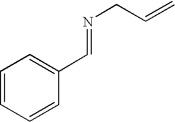 | 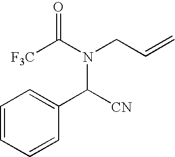 | >99 | 85 |
| 69 | 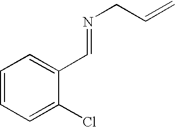 | 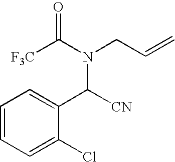 | >99 | 85 |
| 70 | 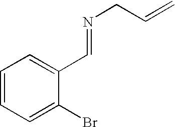 | 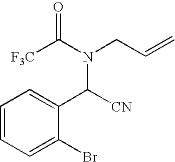 | >99 | 85 |
| 71 | 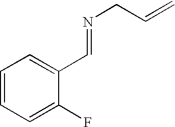 | 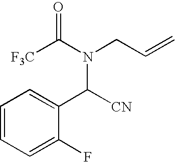 | >99 | 85 |
| 72 | 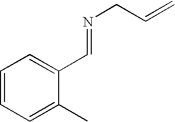 | 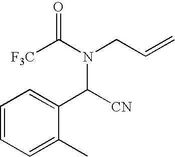 | >99 | 86 |
| 73 | 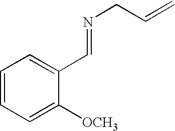 | 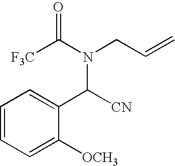 | >99 | 54 |
| 74 | 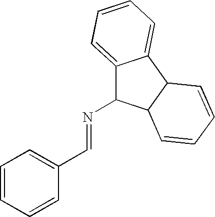 | 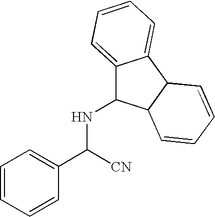 | 98 | 58 |
| 75 | 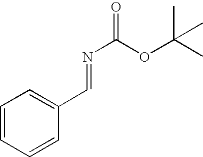 | 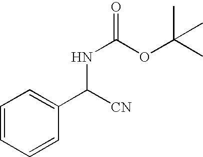 | >99 | >98 |

### Example 76

In the following example, a one-pot asymmetric cyanation reaction was carried out according to the following procedure, as shown in FIG. 3. The chiral titanium catalyst was prepared in situ by stirring the required amount of partially hydrolyzed Ti(On-Bu)₄ in toluene with 200 ppm water for 30 min together with the chiral ligand shown in FIG. 3 (in toluene with 200 ppm water).

In a separate flask, benzaldehyde (0.2 mmol) and benzylamine (0.2 mmol) were stirred for 10-30 min to form the imine in situ. The chiral titanium catalyst (5 mol % based on the aldehyde or amine substrate) and trimethylsilyl cyanide (0.4 mmol) were then added to the flask. The resulting material was stirred at room temperature for 15 minutes, and NMR and HPLC analysis were carried out to determine the yield and enantiomeric excess (ee) of the product. The product was obtained in >99 yield and with an enantiomeric excess of 74%.

What is claimed:

1. A titanium catalyst for asymmetric synthesis reactions, produced by bringing a reaction mixture obtained by contacting water and a titanium alkoxide into contact with an optically active ligand represented by the general formula (a), 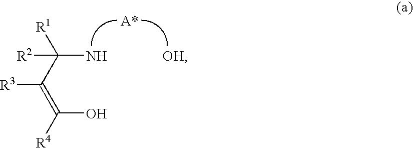
wherein
R¹, R², R³, and R⁴ are independently a hydrogen atom, an alkyl group, an alkenyl group, an aryl group, an aromatic heterocyclic group, a non-aromatic heterocyclic group, an acyl group, an alkoxycarbonyl group or an aryloxycarbonyl group, each of which may have a substituent, or two or more of R¹, R², R³, and R⁴ may be linked together to form a ring, and the ring may have a substituent; and
A* represents a group with two or more carbon atoms having an asymmetric carbon atom or axial asymmetry.

2. The titanium catalyst of claim 1, wherein the optically active ligand represented by said general formula (a) is represented by general formula (b), 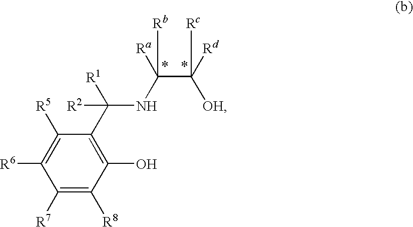
wherein
R^{a}, R^{b}, R^{c}, and R^{d} are each a hydrogen atom, an alkyl group, an aryl group, alkoxycarbonyl group, an aryloxycarbonyl group or an aminocarbonyl group, each of which may have a substituent, or two or more of R^{a}, R^{b}, R^{c}, and R^{d} may be linked together to form a ring, and the ring may have a substituent; at least one of R^{a}, R^{b}, R^{c}, and R^{d} is a different group; both or at least one of the carbon atoms indicated as * become an asymmetric center; and parts indicated as (NH) and (OH) do not belong to A*, and represent an amino group and a hydroxyl group, respectively, corresponding to those in said general formula (a) to which A* is bonded;
R⁵, R⁶, R⁷, and R⁸ are independently a hydrogen atom, a halogen atom, an alkyl group, an alkenyl group, an aryl group, an aromatic heterocyclic group, a non-aromatic heterocyclic group, an alkoxycarbonyl group, an aryloxycarbonyl group, a hydroxyl group, an alkoxy group, an aryloxy group, an amino group, a cyano group, a nitro group, a silyl group or a siloxy group which may have a substituent, each of which may be linked together to form a ring.

3. The titanium catalyst of claim 2, wherein R^{a} is methyl, ethyl, n-propyl, iso-propyl, n-butyl, iso-butyl, sec-butyl, tert-butyl, or benzyl, and R^{b}, R^{c}, and R^{d} are hydrogen atoms.

4. The titanium catalyst of claim 2, wherein the optically active ligand has the structure,

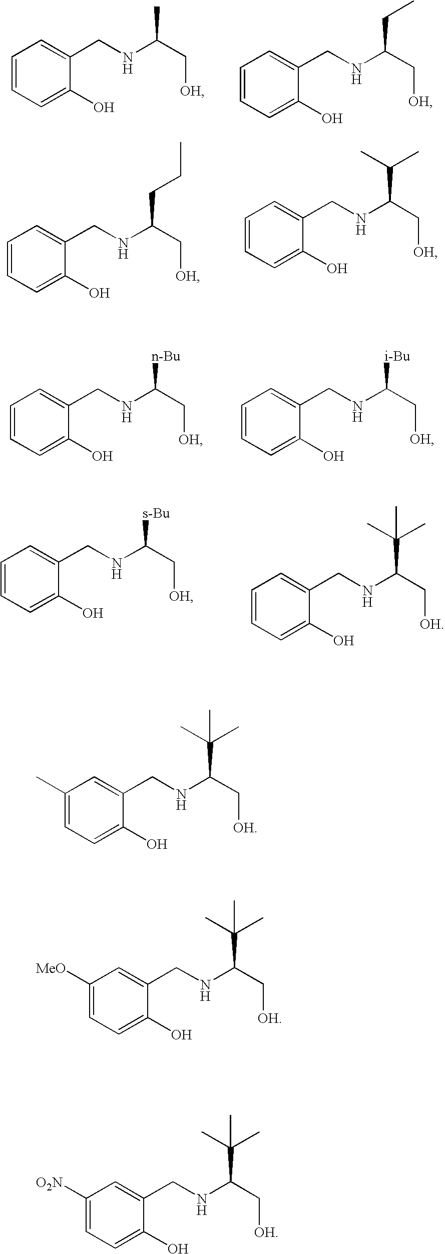

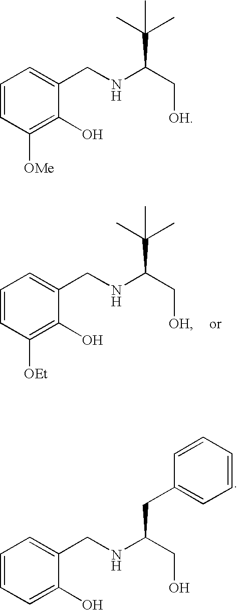

5. A process for asymmetric cyanation of imines, comprising reacting an imine with a cyanating agent in the presence of the titanium catalyst of claim 1.

6. The process for asymmetric cyanation of imines according to claim 5, wherein the process is carried out in presence of an additive having at least one hydroxyl group.

7. The process for asymmetric cyanation of imines according to claim 5, in which said imine is represented by the general formula (c), 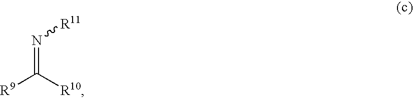
wherein
R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰;
R⁹ and R¹⁰ may be linked together to form a ring, and the ring may have a substituent;
R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl, or sulfoxy group, each of which may have a substituent;
R¹¹ may be linked either to R⁹ or R¹⁰ to form a ring through a carbon chain, and the ring may have substituents.

8. The process for asymmetric cyanation of imines according to claim 5, wherein the cyanating agent is hydrogen cyanide, trialkylsilyl cyanide, acetone cyanohydrin, cyanoformate ester, potassium cyanide-acetic acid, potassium cyanide-acetic anhydride, or tributyltin cyanide.

9. The process for asymmetric cyanation of imines according to claim 5, wherein the cyanating agent is trialkylsilyl cyanide.

10. The process for asymmetric cyanation of imines according to claim 6, wherein the additive is an alcohol, diol, polyol, phenol, or water.

11. The process for asymmetric cyanation of imines according to claims 5, 6, 7, 8, and 9, wherein the imine is generated in situ by reacting a carbonyl compound in presence of a primary amine.

12. The process for asymmetric cyanation of imines according to claim 6, in which said imine is represented by the general formula (c), 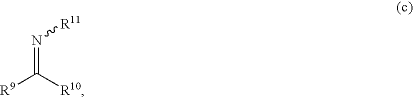
wherein
R⁹ and R¹⁰ are independently a hydrogen atom, an alkyl group, an alkenyl group, an alkynyl group, an aryl group, an aromatic heterocyclic group or a non-aromatic heterocyclic group, each of which may have a substituent, and R⁹ is different from R¹⁰;
R⁹ and R¹⁹ may be linked together to form a ring, and the ring may have a substituent;
R¹¹ is a hydrogen atom, an alkyl group, an alkenyl group, an alkynyl group, an aryl group, an aromatic heterocyclic group or a non-aromatic heterocyclic group, a phosphonate, phosphinoyl, phosphine oxide, alkoxycarbonyl, sulfinyl, or sulfoxy group, each of which may have a substituent; R¹¹ may be linked either to R⁹ or R¹⁰ to form a ring through a carbon chain, and the ring may have substituents.

13. The process for asymmetric cyanation of imines according to claim 6, wherein the cyanating agent is hydrogen cyanide, trialkylsilyl cyanide, acetone cyanohydrin, cyanoformate ester, potassium cyanide-acetic acid, potassium cyanide-acetic anhydride, or tributyltin cyanide.

14. The process for asymmetric cyanation of imines according to claim 6, wherein the cyanating agent is trialkylsilyl cyanide.

15. The process for asymmetric cyanation of imines according to claim 6, wherein the imine is generated in situ by reacting a carbonyl compound in presence of a primary amine.
